(19) United States
(12) Patent Application Publication
(10) Pub. No.: US 2026/0265639 A1
(43) Pub. Date: Sep. 10, 2026

(54) PROCESS FOR PREPARING A SUNFLOWER OLEOSOME COMPOSITION

(71) Applicant: Cargill, Incorporated, Wayzata, MN (US)

(72) Inventors: Eliane Yvonne GOOSSENS, NIEL (BE); Lyndal JENSEN, Wayzata, MN (US); Lucía CABAS-RODRIGUEZ, Almeria (ES)

(73) Assignee: Cargill, Incorporated, Wayzata, MN (US)

(21) Appl. No.: 19/166,175

(22) PCT Filed: Mar. 20, 2024
(86) PCT No.: PCT/US2024/020643

§ 371 Date: Sep. 17, 2025

(30) Foreign Application Priority Data
Mar. 21, 2023 (EP) 23163122.7

(51) Int. Cl. C11B 3/00; A61K 8/92; A61K 47/44; A61Q 19/00

(52) U.S. Cl. CPC C11B 3/008; A61K 8/922; A61K 47/44; A61Q 19/00

(57) ABSTRACT

Present invention relates to process for obtaining a sunflower oleosome composition, comprising steps of: fractionating a sunflower oleosome extract by means of microfiltration, collecting the retentate fraction and obtaining the sunflower oleosome composition; wherein: the sunflower oleosome extract has an amount of total free chlorogenic acids of at least 20000 mg/kg. Moreover, invention relates to a sunflower oleosome composition having an amount total free chlorogenic acids of less than 20000 mg/kg expressed on the dry weight of sunflower oleosome composition; a weight of total lipids of at least 70 wt. % based on the dry weight of the sunflower oleosome composition; and comprising oleosomes, wherein at least 80 wt. % of the total lipids is present in oleosomes. Invention also relates to product comprising the sunflower oleosome composition and to the use of a microfiltration unit to reduce the amount of total chlorogenic acids from a sunflower oleosome extract.

### CROSS-REFERENCE TO RELATED APPLICATIONS

This application claims the benefit of European Patent Application No. 23163122.7, filed 21 Mar. 2023, which is incorporated by reference herein in its entirety.

### FIELD OF THE INVENTION

The present invention relates to a process for preparing a sunflower oleosome composition. The present invention also relates to a sunflower oleosome composition. The present invention further relates to a product comprising the sunflower oleosome composition according to the present invention. It further relates to the use of a microfiltration unit to reduce the amount of total chlorogenic acids (Total CGAs), more preferably chlorogenic acid (CGA).

### BACKGROUND OF THE INVENTION

Oleosomes, also known as “oil bodies”, “lipid bodies”, “lipid droplets” or “spherosomes”, are pre-emulsified droplets or vesicles of oil stored in plant seeds and used as energy sources for plant growth and metabolism. Oleosomes are typically extracted from cells by a process of soaking, washing and grinding the seeds in the presence of water and subsequently filtering or decanting to remove solids and form an aqueous suspension. The suspension is centrifuged to separate the oleosomes, called isolated oleosomes. Once isolated/removed from their natural source, they are known as “isolated oleosomes”.

Unfortunately, the oleosome fractions obtained according to existing processes are of an inferior stability in terms of color and/or taste. This is especially the case when isolating oleosome fractions from sunflower seeds. It is important that the process conditions for isolating oleosome fractions are such that the yield of oleosome fractions obtained from the oil seeds is maximized.

Sunflower oleosomes obtained from sunflower seeds by mild aqueous extraction are obtained as a sunflower oleosome extract. This sunflower oleosome extract in addition comprises proteins and other sunflower seed soluble components such as polyphenol-type compounds and enzymes. Chlorogenic acid (CGA) is the main polyphenol that is found in sunflower oilseeds. It is a reactive component which is prone to oxidation leading to the formation of quinones, which may react further to form green and/or brown pigments. The formation of these pigments limits the use of the sunflower oleosomes in a variety of applications where color and color stability are an essential quality parameter.

Therefore, there is a need in industry to identify an efficient and effective process for obtaining sunflower oleosome fractions for sunflower oleosome compositions having reduced levels of total free CGAs, especially free CGA.

The present invention addresses these issues.

### STATEMENT OF THE INVENTION

In one aspect, the invention relates to a process for obtaining a sunflower oleosome composition, wherein the process comprises the steps of:
i) fractionating by means of microfiltration a sunflower oleosome extract into a retentate fraction and a permeate fraction, and
ii) collecting the retentate fraction, optionally diluting the retentate fraction, and obtaining the sunflower oleosome composition;
further wherein:
   a) the sunflower oleosome extract:
      a1) has a total free CGAs content of at least 20000 mg/kg, preferably an amount of free CGA of at least 15000 mg/kg, based on the dry weight of the sunflower oleosome extract,
      a1) has a weight of total lipids of at least 70 wt. % based on the dry weight of the sunflower oleosome extract, and
      a2) comprises oleosomes, wherein at least 80 wt. % of the total lipids is present in oleosomes; and
   b) the sunflower oleosome composition:
      b1) has a total free CGAs content that is less than the total free CGAs content of a1),
      b2) has a weight of total lipids of at least 70 wt. % based on the dry weight of the sunflower oleosome composition, and
      b3) comprises oleosomes, wherein at least 80 wt. % of the total lipids is present in oleosomes.

In another aspect, the invention relates to a sunflower oleosome composition comprising lipids and proteins, wherein the sunflower oleosome composition:
I. has a total free CGAs content of less than 20000 mg/kg, preferably an amount of free CGA of less than 15000 mg/kg, based on the dry weight of the sunflower oleosome composition,
II. has a weight of total lipids of at least 70 wt. % based on the dry weight of the sunflower oleosome composition, and
III. comprises oleosomes, wherein at least 80 wt. % of the total lipids is present in oleosomes.

In yet another aspect, the invention relates to a product comprising the sunflower oleosome composition, wherein said product is selected from the group consisting of food products, feed products, pharmaceutical products, personal care products, nutritional compositions and industrial products.

Finally, the invention relates to the use of a microfiltration unit to reduce the total amount of total free chlorogenic acids (Total free CGAs), more preferably free chlorogenic acid (CGA) from a sunflower oleosome extract.

Below several preferred features are disclosed. These features are applicable to the process of obtaining sunflower oleosome composition, a sunflower oleosome composition comprising lipids and proteins, a product comprising the sunflower oleosome composition and the use of the sunflower oleosome composition and as well as to the other aspects.

### DESCRIPTION OF FIGURES

FIG. 1: shows the color stability of the oleosome compositions (according to the invention and compared with existing oleosome compositions) by calculation of the difference in absorption at 400 nm and 680 nm between the incubated and initial sample of the aqueous filtrate extracted from the oleosome composition.

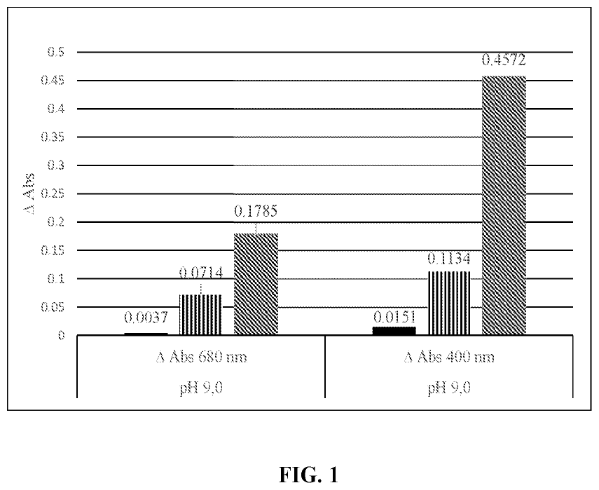

The following legend is used in FIG. 1: Δ Abs for the example according to the invention (solid black); comparative example 1 (vertical stripes); comparative example 2 (diagonal stripes).

### DETAILED DESCRIPTION

The present invention is elucidated below with a detailed description. When used in these specification and claims, the terms “comprises” and “comprising” and variations thereof mean that the specified features, steps or integers are included. The terms are not to be interpreted to exclude the presence of other features, steps or components.

### Total Free Chlorogenic Acids

The aim of the present invention is to reduce from a sunflower oleosome extract the amount of total free CGAs, more preferably the amount of free CGA.

The most predominant polyphenols that are present in sunflower seeds are chlorogenic acid (5-O-caffeoylquinic acid) and its isomeric esters, including its monomers, dimers and polymers. “Total free chlorogenic acids” or “Total free CGAs” is a family of esters formed between quinic acid and certain trans-cinnamic acids, most commonly caffeic, p-coumaric, and ferulic acid. The fact that these total chlorogenic acids are “free”, indicates that the content is measured of the total chlorogenic acids that are not covalently bound with other compounds, such as amongst others proteins. In the present description, the term “Total free chlorogenic acids” or “Total free CGAs” is encompassing 3-O-caffeoylquinic acid (=neochlorogenic acid), 4-O-caffeoylquinic acid (=cryptochlorogenic acid), 5-O-caffeoylquinic acid (chlorogenic acid), 3,4-dicaffeoylquinic acid, 3,5-dicaffeoylquinic acid and 4,5-dicaffeoylquinic acid. From the group of “Total free CGAs”, chlorogenic acid (CGA), also called 5-O-caffeoylquinic acid, is most abundantly present in sunflower seeds.

Kernels of sunflower seeds can comprise up to 4.5 wt. % of polyphenolic compounds, e.g. between 1.9 and 4.2 wt. % of total free chlorogenic acids.

Kernels of sunflower seeds can comprise up to 2 and 3 wt. % of free CGA, making CGA the main component of the total free CGAs to be reduced.

### Process for Obtaining a Sunflower Oleosome Composition

The present invention relates to a process for obtaining a sunflower oleosome composition, wherein the process comprises the steps of:
i) fractionating by means of microfiltration a sunflower oleosome extract into a retentate fraction and a permeate fraction, and
ii) collecting the retentate fraction, optionally diluting the retentate fraction, and obtaining the sunflower oleosome composition;
further wherein:
   a) the sunflower oleosome extract:
      a1) has total free CGAs content of at least 20000 mg/kg, preferably an amount of free CGA of at least 15000 mg/kg, based on the dry weight of the sunflower oleosome extract,
      a2) has a weight of total lipids of at least 70 wt. % based on the dry weight of the sunflower oleosome extract, and
      a3) comprises oleosomes, wherein at least 80 wt. % of the total lipids is present in oleosomes; and
   b) the sunflower oleosome composition:
      b1) has a total free CGAs content that is less than the total free CGAs content of a1),
      b2) has a weight of total lipids of at least 70 wt. % based on the dry weight of the sunflower oleosome composition, and
      b3) comprises oleosomes, wherein at least 80 wt. % of the total lipids is present in oleosomes.

More details regarding the steps i) and ii) of the process according to the invention are disclosed below as well as any additional steps that can be carried out prior to step i), after step ii) or in between step i) and ii).

### Sunflower Oleosome Extract

The starting material in step i) of the process according to the present invention is a sunflower oleosome extract, i.e. an aqueous mixture of seed constituents that have been extracted from sunflower seeds, from which solid particles, such as fibers, have been substantially removed. The constituents extracted from sunflower seeds are amongst others oleosomes and proteins. “Oleosomes”, also known as “oil bodies”, “lipid bodies”, “lipid droplets” or “spherosomes” are pre-emulsified droplets or vesicles that are by nature present in cells or plant seeds for storage of oil. They are used as energy source for plant growth and metabolism.

The sunflower oleosome extract that is used in step i) of the process of the present invention has a total free CGAs content of at least 20000 mg/kg, preferably an amount of free CGA of at least 15000 mg/kg, and weight of total lipids of at least 70 wt. % based on the dry weight of the sunflower oleosome extract. The sunflower oleosome extract comprises oleosomes, wherein at least 80 wt. % of the total lipids is present as oleosomes. The term “dry weight” refers to the weight of the composition, devoid of water. Per definition, “total lipids” relates to the sum of triglycerides (TAGs), diacylglycerides (DAGs), monoacylglycerides (MAGs), free fatty acids (FFAs) and phospholipids. The total lipids is a sum of the lipids present in the oleosomes and the free lipids.

The sunflower oleosome extract in step a1) of the present process may have a total free CGAs content of at least 20000 mg/kg, more than 22000 mg/kg, or more than 23000 mg/kg, such as from 20000 to 145000 mg/kg, from 21000 to 100000 mg/kg based on the dry weight of the sunflower oleosome extract. Preferably, the sunflower oleosome extract in step a1) has a CGA content of more than 15000 mg/kg, more than 18000 mg/kg, more than 20000 mg/kg, such as from 15000 to 40000 mg/kg, or from 20000 to 36000 mg/kg.

Preferably, the sunflower oleosome extract in step a2) of the present process has a weight of total lipids of at least 70 wt. %, at least 75 wt. %, or at least 77 wt. % based on the dry weight of sunflower oleosome extract.

Preferably, the sunflower oleosome extract in step a3) of the present process comprises oleosomes, wherein at least 80 wt. %, at least 85 wt. %, or at least 90 wt. % of the total lipids is present in oleosomes.

“Sunflower” as used in the present description means any type of sunflower seed belonging to the species Helianthus annuus. Several types of sunflower seeds exist, each characterized by the composition of the fatty acid profile of the oil present in these seeds. Regular sunflower seeds contain sunflower oil that is characterized by a typical composition of 66 to 72 wt. % linoleic acid (LA), 9 to 13 wt. % saturated acids, such as palmitic acid (PA) and stearic acid (SA), 17 to 23 wt. % oleic acid (OA), and less than 1 wt. % of alpha-linoleic acid (ALA), expressed on the total weight of fatty acid moiety of the oil. Other well-known varieties of sunflower seeds are so-called mid-oleic (MO) sunflower seeds, high-oleic (HO) sunflower seeds, high oleic-high stearic (HOHS) sunflower seeds, high-palmitic sunflower seeds (HP) and high oleic-high palmitic (HOHP) sunflower seeds, which can be obtained by natural selection or by genetic modification (GMO). Typically, high-oleic sunflower oil is characterized by a weight of 7 to 11 wt. % LA, 7 to 11 wt. % saturated acids (PA and SA), 80 to 86 wt. % oleic acid, and less than 1 wt. % ALA, all expressed on the total weight of fatty acid moiety of the oil. Typically, mid-oleic sunflower oil is characterized by a weight of 24 to 28 wt. % LA, 7 to 11 wt. % saturated acids (PA and SA), 62 to 68 wt. % oleic acid, and less than 1 wt. % ALA, all expressed on the total weight of fatty acid moiety of the oil. The term “fatty acid profile” of a substance, such as an oil, a fat, isolated oleosomes, or an oleosome composition, as used in the present description, mean the total of fatty acids that is present in the oily substance in the form of free fatty acids and in the form of the fatty acid moiety of a lipid (monoglyceride, diglyceride or triglyceride). For example, if an oil is comprising an amount of oleic acid expressed on total weight of the fatty acid profile, this amount is the total of oleic acid present in the oil as a free fatty acid and as oleic acid bound that is bound as the fatty acid moiety in the triglycerides, diglycerides and monoglycerides that are present in the oil.

Preferably, the sunflower seeds used in the process according to the present invention are MO, HO, HOHS, or HOHP sunflower seeds; more preferably HO, HOHS, or HOHP sunflower seeds, most preferably HO sunflower seeds.

The sunflower oleosome extract in step i) of the process of the present invention may have a dry weight of from 5 to 45 wt. %, from 8 to 40 wt. %, or from 10 to 35 wt. %.

The sunflower oleosome extract used in step i) according to the present process may have a pH value in the range of pH 3 to 10, preferably from pH 4 to 9, more preferably from pH 5 to 8 or between 7.2 and 8. Preferably the pH of the sunflower oleosome extract is less than pH 8 to avoid reaction of one or more of the CGAs leading to discoloration (such as a green color in case of CGA) prior to fractionation. In addition, a pH in the range of below pH 8 also assists in avoiding microbial development.

The present invention differs from artificial oleosomes or artificial oil bodies (AOBs) in which a free oil e.g., fish oil is mixed with phospholipids and oleosin protein to form AOBs. The present invention comprises isolated natural oleosomes. The present composition is free of AOBs.

The sunflower oleosome extract may be in the form of an isolated sunflower oleosome extract, a concentrated sunflower oleosome extract, a diluted sunflower oleosome extract or a washed sunflower oleosome extract or mixtures of two or more thereof.

### Obtaining the Sunflower Oleosome Extract Used in Step i)

Methods for obtaining the isolated sunflower oleosome extract that is used in step i) of the process of the present invention are well known in the art. Typically, sunflower plants are grown and allowed to set seed using agricultural cultivation practices well known to a person skilled in the art. The sunflower seeds are harvested and, if desired, materials such as stones or seed hulls (de-hulling) may be removed from the seeds by, for example, sieving or rinsing. Subsequently the sunflower seeds are comminuted (also called grinding) in the presence of an aqueous liquid, e.g. water, into a slurry. The process of comminuting sunflower seeds in the presence of an aqueous liquid is also called wet milling. Following comminuting, solid particles such as fibers are removed from the comminuted slurry by means of centrifugation, decantation or passive filtration over a filter, such as a nylon filter or a cheese cloth. The isolated sunflower oleosome extract is obtained.

Methods for obtaining the isolated sunflower oleosome extract may further comprise a step of roasting the sunflower seeds to a certain degree prior to comminuting in order to avoid green taste of the oleosome composition.

Furthermore, methods for obtaining the isolated sunflower oleosome extract may also comprise a step of soaking the sunflower seeds prior to comminuting.

In one aspect, the isolated sunflower oleosome extract may be concentrated to obtain a concentrated sunflower oleosome extract prior to step i) of the process according to the invention. For concentrating, the isolated sunflower oleosome extract may be centrifuged at a speed sufficient to fractionate the isolated sunflower oleosome extract into a light lipid fraction, comprising most of the oleosomes, i.e. the concentrated sunflower oleosome extract, and a heavy, aqueous fraction.

In another aspect, the isolated sunflower oleosome extract or the concentrated sunflower oleosome extract may be diluted to obtain a diluted sunflower oleosome extract, prior to step i) of the process according to the invention. The sunflower oleosome extract may be diluted by applying a volume of aqueous liquid such that a dilution factor in a range of 2 to 10, 3 to 8, 4 to 6 is obtained. The aqueous liquid may be water, more particular demineralized water, or a buffer, such as a potassium phosphate buffer (e.g. with ionic strength of about 50 mM).

In one more aspect, the isolated sunflower oleosome extract may be washed to obtain a washed sunflower oleosome extract prior to step i) of the process according to the invention. For washing, the isolated sunflower oleosome extract is concentrated and subsequently diluted. The aqueous liquid may be water or a buffer, more particular demineralized water, or a potassium phosphate buffer (e.g. with ionic strength of about 50 mM). The steps of concentrating and subsequently diluting may be applied at least one time, up to 5 times, preferably up to 3 times. A washed sunflower oleosome extract is obtained.

The process steps prior to step i) may be performed in the absence of an organic solvent, such as acetone, hexane, ethanol and the like.

### Process Step i) Fractionation the Sunflower Oleosome Extract by Means of Microfiltration

The fractionation according to step i) of the present process is carried out using microfiltration. Microfiltration is an active filtration process using pressure and a semi-permeable membrane. Microfiltration equipment can, e.g., be based on ceramic membranes (Pall, Tami, Atech), or, polymeric membranes, with membrane housing and pumps conventional in the art.

Step i) of the process according to the invention requires fractionating a sunflower oleosome extract by means of microfiltration. The fractionation step results in a retentate fraction and a permeate fraction. The retentate fraction is the desired fraction of the fractionation process.

The microfiltration according to step i) of the present process is preferably carried out using a membrane having a pore size of less than 2.0 micron, such as less than 0.5 micron. Examples of suitable membrane pore sizes are from 0.01 to 1.0 micron, from 0.05 to 0.5 micron, from 0.1 to 0.2 micron. Using a membrane having pore size of less than 2.0 micron results in a microfiltration step with a good through-put (also called flux) while limiting/avoiding fouling of the pores of the membrane.

The microfiltration according to step i) of the present process may be carried out using a membrane filter that is selected from the group consisting of ceramic membranes, cellulose acetate membranes, polyamides membranes, and polysulphon membranes, and is preferably a ceramic membrane filter.

The microfiltration according to step i) of the present process may be carried out using less than 3 bar, preferably less than 1.5 bar, more preferably less than 1.2 bar, most preferably from 0.7 to 1.15 bar.

The microfiltration according to step i) of the present process may be carried out using a flux in the range of from 10 to 200 kg/h/m² (kilogram per hour per membrane surface area), preferably from 20 to 180 kg/h/m², more preferably from 30 to 50 kg/h/m².

The microfiltration according to step i) of the present process may be carried out at a temperature in the range of 20 to 60° C., preferably 40 to 60° C., more preferably 45 to 55° C. By having a temperature that is around 50° C., it provides control of microbial development.

The microfiltration according to step i) of the present process may be carried out until the dry weight of the retentate fraction has reached a concentration of at most 40 wt. %, at most 30 wt. %, or at most 25 wt. %. Preferably, the microfiltration may be carried out until the dry weight of the retentate fraction has reached a level in a range of from 14 to 35 wt. %, from 17 to 32 wt. %, or from 20 to 27 wt. %. The present inventors have observed that a dry weight within this range results in an efficient microfiltration. The range of dry weight strikes a compromise between the need for excess amounts of an aqueous liquid (e.g. water) and a sufficient flux in the range described above.

Prior to or during the microfiltration, an aqueous solution, such as water, may be added to the sunflower oleosome extract to keep the dry weight to a level of at most 30 wt. %, at most 20 wt. %, or at most 10 wt. %. Preferably, an aqueous solution is added to the sunflower oleosome extract prior or during the microfiltration according to step i) of the present invention to keep the dry weight in a range of from 5 to 30 wt. %, from 8 to 25 wt. %, or from 10 to 15 wt. %.

The microfiltration is preferably carried out in such a manner that ultrasound microturbulence may be applied to avoid fouling of the membranes.

### Optional Steps in Between Step i) and Step ii)

The retentate fraction obtained in step i) may be subjected to one or more further fractionations by means of microfiltration according to step i) of the present invention. The one or more further fractionation steps may result into one or more further retentate fractions and one or more further permeate fractions, and wherein said one or more further retentate fractions are collected. Optionally the one or more further retentate fraction may be diluted to obtain the sunflower oleosome composition. This step is carried out prior to the collection of the (further) retentate fraction as the sunflower oleosome composition. This optional step is in fact one or more steps of recycling to further reduce the total CGAs concentration prior to obtaining the composition according to the invention.

### Process Step ii)

After fractionation by means of microfiltration in step i) according to the present invention (and optionally one or more recycling steps), a (further) retentate fraction is collected in step ii) as the sunflower oleosome composition.

The obtained sunflower oleosome composition may have a dry weight of about 20-40 wt. %.

Optional Steps after Step ii)

After the retentate fraction (or further retentate fraction) is obtained in step ii) one or more optional steps may be carried out such as dilution, heat-treatment, dehydration or a combination thereof. These steps are discussed in some detail below.

The sunflower oleosome composition may be diluted with an aqueous solution, such as water. A diluted sunflower oleosome composition may be obtained. The diluted sunflower oleosome composition may have a lipid content in a range of from 0.5 to 5.0 wt. %, from 0.8 to 4.0 wt. %, or from 1.0 to 3.0 wt. %, expressed on total weight of the composition, wherein at least 85 wt. %, at least 90 wt. %, or at least 92 wt. % of the total lipids is present in oleosomes. The protein content of the diluted sunflower oleosome composition may be in a range of from 0.1 to 2.14, from 0.2 to 2.0, or from 0.3 to 1.8 wt. %, expressed on total weight of the composition.

The sunflower oleosome composition may be subjected to a heat treatment step. The heat treatment may be a pasteurization treatment or an ultra-high-temperature (UHT) treatment. Pasteurization treatment involves heating the sunflower oleosome composition at 65° C. to 70° C. for 30 minutes in batch, preferably 72° C. to 86° C. for about 30 seconds in a continuous-flow process ((High-temperature short time Pasteurization (HTST Pasteurization)). UHT treatment involves heating of the sunflower oleosome composition at a temperature of 138° C. to 150° C. in a continuous-flow process and holding at that temperature for one or more seconds, up to 5 seconds, before cooling rapidly to room temperature. The heat treatment step of the sunflower oleosome composition is applied to further avoid microbial contamination of the sunflower oleosomes.

In an aspect, the present process comprises the steps of:
step i) fractionating by means of microfiltration a sunflower oleosome extract into a retentate fraction and a permeate fraction;
step ii) collecting the retentate fraction, optionally diluting the retentate fraction, and obtaining the sunflower oleosome composition; and
heat treating the sunflower oleosome composition to obtain a heat-treated sunflower oleosome composition.

Following any of the steps mentioned above, the oleosome composition may be subjected to a dehydration step. Dehydration is well known to the person skilled in the art such as amongst others spray drying, fluid bed drying, freeze-drying, and vacuum drying. In one aspect of the invention, the dehydration step is a spray-drying step.

In an aspect, the present process comprises, after step ii) the steps of:
dehydrating the heat-treated sunflower oleosome composition by spray-drying to obtain a spray-dried heat-treated washed sunflower oleosome composition.

Following the fractionation step, the sunflower oleosomes of the sunflower oleosome composition may be subjected to an enlargement step. The enlarging may be carried out by applying high-shear centrifugation force to the sunflower oleosome composition and/or a process of applying high-shear mixing to the sunflower oleosome composition, such as described in WO2021126408A1 by the present applicant.

Following the fractionation step (or after the enlargement step), the oleosomes or enlarged oleosomes may be subjected to a loading step with one or more source of alpha-linolenic acid (ALA) and/or long-chain poly-unsaturated fatty acids (LC-PUFA) and/or medium-chain fatty acids (MCFA) and/or triglyceride having saturated C16 fatty acids that are positioned at the sn2 position and/or lipophilic bioactive substances. The step of loading may be carried out by first blending the oleosome composition with the one or more source in a ratio of source to oleosomes of from 1:99 to 95:5; and subjecting the blend obtained to a high-shear force, preferably followed by high-pressure homogenization. Alternatively, the high-shear force can be replaced by ultrasound.

Isolated sunflower oleosomes that have been loaded with one or more additional components, such as lipids or lipophilic bioactive substances, which components are encapsulated by the sunflower oleosomes, thus present in the inside thereof, are called loaded isolated sunflower oleosomes.

Lipophilic dietary bioactive substances may be selected from the group consisting of oil-soluble vitamins, phytosterols, curcuminoids, carotenoids, and flavonoids, and combinations of two or more thereof.

### Sunflower Oleosome Composition

The present invention also relates to a sunflower oleosome composition comprising lipids and proteins, wherein the sunflower oleosome composition:
I. has a total free CGAs content of less than 20000 mg/kg, preferably an amount of free CGA of less than 15000 mg/kg, based on the dry weight of the sunflower oleosome composition,
II. has a weight of total lipids of at least 70 wt. % based on the dry weight of the sunflower oleosome composition, and
III. comprises oleosomes, wherein at least 80 wt. % of the total lipids is present in oleosomes.

### Feature I—Total CGA Concentration of the Sunflower Oleosome Composition

The present sunflower oleosome composition has a has a total free CGAs content of less than 20000 mg/kg, preferably has a total free CGAs content of less than 5000 mg/kg, has a total free CGAs content of less than 700 mg/kg, such as from 0.1 to 600 mg/kg, expressed on the dry weight of the sunflower oleosome composition.

Preferably, the sunflower oleosome composition has a free CGA content of less than 15000 mg/kg, less than 4000 mg/kg, less than 500 mg/kg, such as in a range of from 0.05 to 400 mg/kg, expressed on the dry weight of the sunflower oleosome composition.

### Feature II—Total Weight of Lipids of the Sunflower Oleosome Composition

Preferably, the weight of total lipids in the sunflower oleosome composition may be at least 75 wt. %, at least 80 wt. %, or at least 85 wt. % based on the total dry weight of the sunflower oleosome composition. For example, the amount of total lipids in the sunflower oleosome composition may be between 75 and 90 wt. %, from 78 to 88 wt. %, based on the total dry weight of the sunflower oleosome composition.

“Weight total lipid” as used in the present description means the dry weight of all the lipids present in the composition, thus both present in the oleosomes (in the center as well as in the membrane surrounding the oleosome) and outside of the oleosomes, the latter being called “free lipids”.

### Feature III—Total Weight of Lipids Present as Oleosomes

Preferably, the weight of lipids that is present in oleosomes (Feature III) is at least 85 wt. %, at least 90 wt. %, or at least 92 wt. % of the total lipids. It is preferred that substantially all, or all of the lipids is present in sunflower oleosomes. The remaining weight of lipids, i.e. the weight of lipids that is not present in the sunflower oleosomes expressed on the weight of total lipids that are present in the sunflower oleosome composition, may be present in the composition in free form, i.e. outside of the oleosomes. In other words, the composition then comprises free lipids in addition to lipids that are present inside oleosomes. In an aspect, substantially all lipids or at least 95% are present inside oleosomes for optimal protection of these lipids. The combination of lipids that are present in the oleosomes and the free lipids thus adds up to 100 wt. % based on the weight total lipid in the sunflower oleosome composition.

The weight of free lipids in the sunflower oleosome composition can be quantified by extracting the free lipids from the sunflower oleosome composition with heptane. The heptane will only extract the free lipids, not the lipids in the oleosomes. Subsequently the weight of lipids in the heptane phase is quantified. Quantification can be done by means of GPC analysis.

The weight of lipids in the sunflower oleosome composition that is present in the oleosomes is calculated as the difference between the weight total lipid (for example measured by Soxhlet method) and the weight of free lipids in the sunflower oleosome composition.

### Total Weight of Protein of the Sunflower Oleosome Composition

The sunflower oleosome composition may have a total weight of protein of up to 25 wt. %, preferably up to 20 wt. %, or up to 15 wt. % based on the dry weight of the oleosome composition. With the “total protein” content is meant all protein that is present in said composition. The amount of total protein may be at least 5 wt. %, or at least 10 wt. %.

Oleosomes are comprising intrinsic proteins such as mainly oleosin and minor amounts of caleosin and steroleosin. Without wishing to be bound by a particular theory, the present inventors observed that the oleosins contain a hydrophilic part, which is present at the isolated oleosomes' surface, and a hydrophobic part which is anchored in the oil in the center of the oleosomes and ensures for oleosome stability. Even at alkaline conditions of pH 8 or higher, intrinsic proteins remain strongly bound, whereas weakly bound proteins will be removed in alkaline conditions. Isolated oleosomes may have a content of intrinsic proteins in an amount of from 0.2 to 6.0 wt. % based on the dry weight of the isolated oleosomes, such as from 0.3 to 5.5 wt. %, or from 0.3 to 5.2 wt. %. Generally, they are present in an amount of approx. 3 wt. %. The content of the intrinsic proteins is measured after washing oleosomes at pH 9.5. The applied method is described in the experimental section in paragraphs [0117]-[0120] of WO2021126408A1 which section is incorporated by reference.

In addition to intrinsic oleosome proteins, also other sunflower-seed proteins are present in the composition. Preferably, the amount of total protein is high. Other ways to reduce the content of total CGAs that are existing in the art, involve washing the oleosomes. When washing the content of total CGAs will be reduced but also a large amount of proteins and even oleosomes will be removed from the oleosome composition. Loss of proteins and oleosomes means a loss of yield which is undesirable. A high amount of proteins is beneficial for the nutritional value of the oleosomes.

The amount of protein in the composition can also be specified in relation to the amount of total CGA that is present. The weight ratio of total protein to total CGA may be in the range of from 20/1 to 5/1, from 12/1 to 9/1.

The sunflower oleosome composition may be in liquid form, e.g. as obtained after fractionation and collection of the retentate fraction. However, as discussed above the sunflower oleosome composition may also be in heat-treated form, e.g. pasteurized or UHT-treated. The sunflower oleosome composition may also be in dehydrated (dried) form, e.g. in spray-dried form or in freeze-dried form.

### Color of the Sunflower Oleosome Composition

The measurement of color of the oleosome composition is interfered by the concentration of the oleosomes present in liquid form. Therefore, the color stability of the oleosome composition is determined by an indirect method, using the filtrate of the oleosome composition.

A covalent or non-covalent interaction between co-extracted proteins and CGA was induced by incubating the oleosome composition in liquid form for 24 h at pH 9 (covalent) or pH 7 (non-covalent) using 1 M of NaOH or 1 M of HCl, followed by filtrating off the oleosomes. It is important to highlight that all color reactions (e.g. CGA-protein complexation) take place in the aqueous phase. Absorbances (Abs) between 400 and 800 nm (visible range) were detected. The color stability was expressed as the difference in absorption at 400 nm and 680 nm between the initial sample and the incubated sample according to the following formula:

$${ΔAbs}_{i n c u b ated - initial} = {Abs}_{i n c u b a t e d} - {Abs}_{initial}$$

A lower ΔAbs value represents a more stable color. Wavelengths at 400 and 680 nm represent the color absorption peaks for violet and orange, respectively. The complementary colors for 400 and 680 nm are yellow and blue, which in fact are the ones that can be visualized by the human eye

The oleosome composition shows a difference in absorption at 680 nm and at pH 7 of from 0.0100 to 0.0280, or from 0.02 to 0.027.

The oleosome composition shows a difference in absorption at 400 nm and at pH 7 of from 0.0200 to 0.0450, or from 0.03 to 0.043.

The oleosome composition shows a difference in absorption at 680 nm and at pH 9 of from 0.00100 to 0.060, or from 0.002 to 0.04.

The oleosome composition shows a difference in absorption at 400 nm and at pH 9 of from 0.0100 to 0.1, or from 0.02 to 0.07.

In an aspect, the sunflower oleosome composition does not contain acidulants and/or reducing agents.

### Products Comprising the Sunflower Oleosome Composition

The invention also relates to products comprising the oleosome composition, wherein the product is selected from the group consisting of food products, feed products, pharmaceutical products, personal care products, nutritional compositions, and industrial products.

Furthermore, the current invention relates to a process for preparing the product according to the invention comprising the sunflower oleosome composition; the process comprising the step of blending the sunflower oleosome composition with the at least one other ingredient other than the sunflower oleosome composition. The process preferably does not comprise a further step of emulsification of the sunflower oleosome composition with the at least one other nutritional ingredient.

The product comprising the sunflower oleosome composition according to the invention, may be a nutritional composition comprising the sunflower oleosome composition in a range of from 0.1 to 70.0 wt. %, preferably between 2.0 and 50.0 wt. % on dry weight of the nutritional composition. For example, in an amount of from 5.0 to 65.0 wt. %, from 9.3 to 60.0 wt. %, or from 20.6 to 55.0 wt. % on dry weight of the nutritional composition. More specifically in an amount of from 0.2 to 22.0 wt. %, such as 0.3 wt. %, 3.0 wt. %, 10.0 wt. %, 21.0 wt. %. The nutritional composition is further comprising at least one additional nutritional ingredient, other than sunflower oleosome composition. The at least one nutritional ingredient is not derived from sunflower oleosome composition. Nutritional ingredients are ingredients that contribute to the caloric intake and/or provide micronutrients. With “on dry weight of the nutritional composition” is meant that it is expressed on weight of all components except water.

Specifically, the present invention relates to the product comprising the oleosome composition being the nutritional composition in spray dried form.

The additional nutritional ingredient may be selected from the group consisting of sources of proteins, sources of fats, sources of carbohydrates, sources of micronutrients, and combinations of two or more thereof. The term “micronutrients” is encompassing nutrients that an organism needs in small quantities for the proper functioning of its metabolism. Examples of micronutrients are, but are not limited to vitamins, minerals, trace elements, essential amino acids and essential fatty acids.

The nutritional composition may further comprise at least one non-nutritional ingredient. Non-nutritional ingredients according to the invention are ingredients that do not substantially add to the caloric intake and/or do not substantially provide micronutrients. Examples of non-nutritional ingredients are flavors, colorants, emulsifiers, acid regulators such as citric acid or lactic acid, preservatives, and the like. The non-nutritional ingredients may be from a natural or synthetic origin.

In the nutritional composition the combination of the sunflower oleosome composition, the at least one additional nutritional ingredient and optionally water and/or non-nutritional ingredients make up 100 wt. %.

Examples of nutritional compositions according to the present invention may be compositions that are developed to cover the nutritional needs, either as a supplement or as complete nutrition. The people that are targeted for the nutritional composition according to the invention relate to specific groups of people, such as, but not limited to, preterm infants, infants, toddlers, elderly people, pregnant women, athletes, or humans having nutritional deficiencies and/or having a deficient immune system. In a specific aspect, the nutritional composition is an infant formula. This infant formula may be milk-based, e.g. with milk protein isolate and/or caseinate and/or whey protein, or it may be plant-based, e.g. with soy, rice, corn and/or pea proteins. In a specific aspect, the nutritional composition is for pregnant women. The nutritional compositions may be designed for people suffering from a more specific disease state such as cancer, chronic obstructive pulmonary disease, and later-stage kidney disease, and others. Amongst others, nutritional compositions may be helpful for people who struggle with a loss of appetite, have difficulty with chewing, have trouble preparing balanced meals, and/or are recovering from surgery or an illness. If the nutritional composition is meant for complete nutrition, it can provide a healthy balance of protein, carbohydrate, and/or fat.

These nutritional compositions can be in the form of liquid, as a ready-to-drink formula, or used in feeding tubes. It can also be in the form of a formula base i.e., a powder or a concentrated liquid, to be dissolved in water or another fluid for the preparation of a ready-to-drink nutritional composition. The nutritional composition may also be in the form of a pudding or a jelly, or the form of a cookie or a snack bar, or any other form.

In yet another aspect of the invention, the at least one nutritional ingredient other than oleosomes is not an emulsifier.

In one specific aspect, the nutritional composition according to the invention comprising sunflower oleosome composition is a food product, such as infant formula, preferably growing-up milk. The infant food product or infant formula is a term well-known in the art and it refers to food that is specifically manufactured for infants and it may be characterized in that is soft, and easily consumable by infants and has a nutritional composition adapted to the specific needs at each growth stage.

In another specific aspect, the nutritional composition according to the invention comprising the sunflower oleosome composition is a nutritional composition, such as a nutritional drink, a nutritional powder, such as a sport nutrition powder; a powder for food fortification, a nutritional bar or cookie, such as a sports nutrition bar; or a nutritional supplement.

Examples of other types of food and feed products include but are not limited to drinks such as coffee, black tea, powdered green tea, cocoa, and juice; milk component-containing drinks, such as raw milk, processed milk, and lactic acid drinks; a variety of drinks including nutrition-enriched drinks, such as calcium-fortified drinks and the like and dietary fiber-containing drinks; dairy products, such as butter, cheese, vegan cheese, yoghurt, coffee whitener, whipping cream, custard cream, and custard pudding; iced products, sherbet, and frozen yogurt; processed fat food products, such as mayonnaise, margarine, spread, and shortening; soups; stews; seasonings such as sauce and dressings; a variety of paste condiments represented by kneaded mustard; a variety of fillings typified by jam and flour paste; a variety or gel or paste-like food products including red bean-jam, jelly, and foods for swallowing impaired people; food products containing cereals as the main component, such as bread, noodles, pasta, pizza pie, and corn flake; Japanese, US and European cakes, candy, cookie, biscuit, hot cake, chocolate, and rice cake; kneaded marine products represented by (boiled) fish cake; live-stock products represented by ham, sausage, hamburger and steak; daily dishes such as cream croquette, paste for Chinese foods, gratin, and dumpling; foods of delicate flavor, such as salted fish guts and a vegetable pickled in sake lee; liquid diets such as tube feeding liquid food; supplements; and pet foods. Whereas traditional processes for preparing food and feed products will require an emulsification or homogenization step, the process of the current invention for preparing the food and feed products does not need such a homogenization or emulsification step. A simple blending of the sunflower oleosome composition with the other ingredients is sufficient.

Examples of such pharmaceutical products include products comprising therapeutic agents, diagnostic agents, and delivery agents. As a therapeutic or diagnostic agent, the product will additionally contain an active ingredient. The active ingredient can be anything that one wishes to deliver to a host. The active ingredient may be a protein or peptide that has therapeutic or diagnostic value. Such peptides include antigens (for vaccine formulations), antibodies, cytokines, blood-clotting factors, and growth hormones. An example of a pharmaceutical product is a parenteral emulsion containing the sunflower oleosome composition and a drug.

Examples of such personal care products include soaps, cosmetics, skin creams, facial creams, toothpaste, lipstick, perfumes, make-up, foundation, blusher, mascara, eyeshadow, sunscreen lotions, hair conditioner, and hair coloring.

Examples of such industrial products include paints, coatings, lubricants, films, gels, drilling fluids, paper sizing, latex, building, and road construction material, inks, dyes, waxes, polishes, and agrochemical formulations.

### Use of a Microfiltration Unit According to the Invention

As discussed above, the present invention relates to a process for obtaining a sunflower oleosome composition and said composition comprising lipids and proteins.

The present invention further relates to the use of a microfiltration unit to reduce the amount of total free chlorogenic acids (Total Free CGAs), more preferably free chlorogenic acid (CGA) from a sunflower oleosome extract.

It relates to the use wherein the content of total free CGAs is less than 20000 mg/kg, preferably the total free CGAs content is less than 5000 mg/kg, a total free CGAs content of less than 700 mg/kg, such as from 0.1 to 600 mg/kg, expressed on the dry weight of the sunflower oleosome composition.

It relates to the use wherein the content of free CGA is in a range of less than 15000 mg/kg, less than 4000 mg/kg, less than 500 mg/kg, such as in a range of from 0.05 to 400 mg/kg, expressed on the dry weight of the sunflower oleosome composition.

It relates to the use of a microfiltration unit to reduce the amount of total free chlorogenic acids (Total Free CGAs), more preferably free chlorogenic acid (CGA) from a sunflower oleosome extract wherein
a) the sunflower oleosome extract:
   a1) has a total free CGAs content of at least 20000 mg/kg, preferably an amount of free CGA of at least 15000 mg/kg, based on the dry weight of the sunflower oleosome extract,
   a2) has a weight of total lipids of at least 70 wt. % based on the dry weight of the sunflower oleosome extract, and
comprises oleosomes, wherein at least 80 wt. % of the total lipids is present in oleosomes.

It further relates to the use wherein a sunflower oleosome composition is obtained and:
b) the sunflower oleosome composition:
   b1) has a total free CGAs content that is less than the total free CGAs content of a1),
   b2) has a weight of total lipids of at least 70 wt. % based on the dry weight of the sunflower oleosome composition, and
   b3) comprises oleosomes, wherein at least 80 wt. % of the total lipids is present in oleosomes.

It further relates to the use of microfiltration unit wherein a sunflower oleosome composition is obtained and the sunflower oleosome composition has a content of total free CGAs of from 0.1 to 600 mg/kg and a content of free CGA in a range of from 0.05 to 400 mg/kg expressed on the dry weight of the sunflower oleosome composition.

It further relates to the use of microfiltration unit wherein a sunflower oleosome composition is obtained and the sunflower oleosome composition has a content of total free CGAs of from 0.1 to 300 mg/kg and a content of free CGA in a range of from 0.1 to 150 mg/kg expressed on the dry weight of the sunflower oleosome composition.

### Effects of the Invention

The present invention relates to a sunflower oleosome composition that is low in total free CGA, preferably free CGA. This has the beneficial effect that the sunflower oleosome composition will not turn green. There is no need for the addition of an acidulant or reducing agent to suppress or mask the green color, since the total CGA content has been reduced.

Other prior art methods for removing CGA by means of washing the sunflower oleosome composition, may additionally lead to removal of proteins and optionally oleosomes. The present invention allows to achieve higher yield and higher protein to lipid ratios while at the same time preventing (reducing) green color. One of the beneficial effects of the increased protein to lipid ratio is the improved nutritional value of the oleosome composition. The removal of the total CGA is a more effective approach and since it not necessary to add additives, it is a more cost-effective approach as well as a cleaner approach leading to products having less added ingredients.

### CLAUSES

1. A sunflower oleosome composition comprising lipids and proteins, wherein the sunflower oleosome composition:
I. has a total free CGAs content of less than 20000 mg/kg, based on the dry weight of the sunflower oleosome composition,
II. has a weight of total lipids of at least 70 wt. % based on the dry weight of the sunflower oleosome composition, and
III. comprises oleosomes, wherein at least 80 wt. % of the total lipids is present in oleosomes.

2. The sunflower oleosome composition according to clause 1 wherein the content of total free CGAs is less than 5000 mg/kg based on the dry weight of the sunflower oleosome composition.

3. The sunflower oleosome composition according to clause 1 or 2 wherein the content of total free CGAs is less than 2500 mg/kg based on the dry weight of the sunflower oleosome composition.

4. The sunflower oleosome composition according to anyone of clauses 1 to 3 wherein the content of total free CGAs is less than 1000 mg/kg based on the dry weight of the sunflower oleosome composition.

5. The sunflower oleosome composition according to anyone of clauses 1 to 4 wherein the content of total free CGAs is less than 700 mg/kg based on the dry weight of the sunflower oleosome composition.

6. The sunflower composition according to anyone of clauses 1 to 5 wherein the content of total free CGAs is less than 650 mg/kg based on the dry weight of the sunflower oleosome composition.

7. The sunflower composition according to anyone of clauses 1 to 6 wherein the content of total free CGAs is from 0.1 to 600 mg/kg based on the dry weight of the sunflower oleosome composition.

8. The sunflower composition according to anyone of clauses 1 to 7 wherein the content of total free CGAs is from 0.1 to 300 mg/kg, expressed on the dry weight of the sunflower oleosome composition.

9. The sunflower composition according to anyone of clauses 1 to 8 wherein the content of free CGA is less than 15000 mg/kg expressed on the dry weight of the sunflower oleosome composition.

10. The sunflower composition according to anyone of clauses 1 to 9 wherein the content of free CGA is less than 4000 mg/kg expressed on the dry weight of the sunflower oleosome composition.

11. The sunflower composition according to anyone of clauses 1 to 10 wherein the content of free CGA is less than 500 mg/kg expressed on the dry weight of the sunflower oleosome composition.

12. The sunflower composition according to anyone of clauses 1 to 11 wherein the content of free CGA is in a range of from 0.05 to 400 mg/kg expressed on the dry weight of the sunflower oleosome composition.

13. The sunflower composition according to anyone of clauses 1 to 12 wherein the content of free CGA is from 0.1 to 150 mg/kg expressed on the dry weight of the sunflower oleosome composition.

14. The sunflower oleosome composition according to anyone of clauses 1 to 13 wherein the content of total free CGAs is from 0.1 to 600 mg/kg and the content of free CGA is a range of from 0.05 to 400 mg/kg expressed on the dry weight of the sunflower oleosome composition.

15. The sunflower oleosome composition according to anyone of clauses 1 to 14 wherein the content of total free CGAs is from 0.1 to 300 mg/kg and the content of free CGA is a range of from 0.1 to 150 mg/kg expressed on the dry weight of the sunflower oleosome composition.

16. The sunflower oleosome composition according to anyone of clauses 1 to 15 wherein the sunflower oleosome composition has a weight of total lipids of at least 70 wt. % based on the dry weight of the sunflower oleosome composition.

17. The sunflower oleosome composition according to anyone of clauses 1 to 16 wherein the sunflower oleosome composition has a weight of total lipids of at least 75 wt. % based on the dry weight of the sunflower oleosome composition.

18. The sunflower oleosome composition according to anyone of clauses 1 to 17 wherein the sunflower oleosome composition has a weight of total lipids of at least 80 wt. % based on the dry weight of the sunflower oleosome composition.

19. The sunflower oleosome composition according to anyone of clauses 1 to 18 wherein the sunflower oleosome composition has a weight of total lipids of at least 85 wt. % based on the dry weight of the sunflower oleosome composition.

20. The sunflower oleosome composition according to anyone of clauses 1 to 19 wherein the sunflower oleosome composition has a weight of total lipids of at least 87 wt. % based on the dry weight of the sunflower oleosome composition.

21. The sunflower oleosome composition according to anyone of clauses 1 to 20 wherein at least 80 wt. % of the total lipids is present in oleosomes.

22. The sunflower oleosome composition according to anyone of clauses 1 to 21 wherein at least 85 wt. % of the total lipids is present in oleosomes.

23. The sunflower oleosome composition according to anyone of clauses 1 to 22 wherein at least 90 wt. % of the total lipids is present in oleosomes.

24. The sunflower oleosome composition according to anyone of clauses 1 to 23 wherein at least 93 wt. % of the total lipids is present in oleosomes.

25. The sunflower oleosome composition according to anyone of clauses 1 to 24 wherein from 93 to 98 wt % of the total lipids is present in oleosomes.

26. The sunflower oleosome composition according to anyone of clauses 1 to 25, wherein the sunflower oleosome composition:
I. has content of total free CGAs from 0.1 to 600 mg/kg, expressed on the dry weight of the sunflower oleosome composition,
II. content of free CGA is a range of from 0.05 to 400 mg/kg expressed on the dry weight of the sunflower oleosome composition.

27. The sunflower oleosome composition according to anyone of clauses 1 to 26, wherein the sunflower oleosome composition:
I. has content of total free CGAs from 0.1 to 600 mg/kg, expressed on the dry weight of the sunflower oleosome composition,
II. content of free CGA is a range of from 0.05 to 400 mg/kg expressed on the dry weight of the sunflower oleosome composition,
III. has weight of total lipids of at least 87 wt. % based on the dry weight of the sunflower oleosome composition, and
IV. comprises oleosomes, wherein from 93 to 98 wt % of the total lipids is present in oleosomes.

28. The sunflower oleosome composition according to anyone of clauses 1 to 27, wherein the sunflower oleosome composition:
I. has content of total free CGAs from 0.1 to 300 mg/kg, expressed on the dry weight of the sunflower oleosome composition,
II. content of free CGA is a range of from 0.1 to 150 mg/kg expressed on the dry weight of the sunflower oleosome composition,
III. has weight of total lipids of at least 87 wt. % based on the dry weight of the sunflower oleosome composition, and
IV. comprises oleosomes, wherein from 93 to 98 wt % of the total lipids is present in oleosomes.

29. A product comprising the sunflower oleosome composition according to any one of clauses 1 to 28, wherein said product is selected from the group consisting of food products, feed products, pharmaceutical products, personal care products, nutritional compositions and industrial products.

30. A product selected from the group consisting of food products, feed products, pharmaceutical products, personal care products, nutritional compositions and industrial products, and the product is prepared by adding the sunflower oleosome composition according to any one of clauses 1 to 28 to product ingredients that are not a sunflower oleosome composition.

31. A process for obtaining a sunflower oleosome composition, wherein the process comprises the steps of:
i) fractionating by means of microfiltration a sunflower oleosome extract into a retentate fraction and a permeate fraction, and
ii) collecting the retentate fraction, optionally diluting the retentate fraction, and obtaining the sunflower oleosome composition;
further wherein:
   a) the sunflower oleosome extract:
      a1) has a total free CGAs content of at least 20000 mg/kg, preferably an amount of free CGA of at least 15000 mg/kg, based on the dry weight of the sunflower oleosome extract,
      a2) has a weight of total lipids of at least 70 wt. % based on the dry weight of the sunflower oleosome extract, and
      a3) comprises oleosomes, wherein at least 80 wt. % of the total lipids is present in oleosomes; and
   b) the sunflower oleosome composition:
      b1) has a total free CGAs content of less than 700 mg/kg and a free CGA of less than 500 mg/kg;
      b2) has a weight of total lipids of at least 70 wt. % based on the dry weight of the sunflower oleosome composition, and comprises oleosomes, wherein at least 80 wt. % of the total lipids is present in oleosomes.

32. The process according to clause 31 for obtaining a sunflower oleosome composition, wherein the process comprises the steps of:
iii) fractionating by means of microfiltration a sunflower oleosome extract into a retentate fraction and a permeate fraction, and
iv) collecting the retentate fraction, optionally diluting the retentate fraction, and obtaining the sunflower oleosome composition;
further wherein:
   a) the sunflower oleosome extract:
      a1) has a total free CGAs content of at least 20000 mg/kg, preferably an amount of free CGA of at least 15000 mg/kg, based on the dry weight of the sunflower oleosome extract,
      a2) has a weight of total lipids of at least 70 wt. % based on the dry weight of the sunflower oleosome extract, and
      a3) comprises oleosomes, wherein at least 80 wt. % of the total lipids is present in oleosomes; and
   b) the sunflower oleosome composition:
      b1) has a total free CGAs content of 0.1 to 300 mg/kg and a free CGA of from 0.1 to 150 mg/kg;
      b2) has a weight of total lipids of at least 70 wt. % based on the dry weight of the sunflower oleosome composition, and
         comprises oleosomes, wherein at least 80 wt. % of the total lipids is present in oleosomes.

Although certain aspects of the invention have been described, the scope of the appended claims is not intended to be limited solely to these specific aspects. The claims are to be construed literally, purposively, and/or to encompass equivalents. The scope of the present invention is defined by the appended claims. One or more of the objects of the invention are achieved by the appended claims.

### EXAMPLES

The present invention is further elucidated based on the Examples below which are illustrative only and not considered limiting to the present invention.

### 1. Analysis Methods

### Protein Measurement

The protein content of the oleosomes was determined by the amount of Nitrogen in the sample. This amount of Nitrogen was analyzed using a combustion method according to Dumas. Combustion of the sample was performed at 1100° C. The amount of Nitrogen was determined using a conductivity detector (LECO TruMAc).

The protein content was calculated by multiplying the amount of Nitrogen analyzed by 6.25.

### Measurement of the Lipid Content

The weight of free lipids can be measured as well as the weight of total lipids. The difference between these values leads to the weight of lipids that is present inside of the oleosomes.

### Measurement of Free Lipid

400 mg of sunflower oleosome sample was mixed with 7 mL heptane in a capped glass test tube and subjected to ultrasonication for 10 min for extraction of the free oil into the heptane phase. After extraction the solution is centrifuged at 4500 rpm (3283× g) for 5 minutes followed by filtration of the top heptane layer through a 0.45 μm PTFE syringe filter (Merck). The extracted free oil was quantified by gel permeation chromatography (GPC) on an Alliance HPLC system from Waters on two GPC columns placed in series (Styragel HR 1, 100 Å, 5 μm, 7.8 mm×300 mm in DMF, 100-5K, 1/pk; Styragel HR 0.5, 50 Å, 5 μm, 7.8 mm×300 mm in THF, 0-1K, 1/pk Waters HR 0.5, Waters) running isocratically with 2-methyl-tetrahydrofuran at a flow of 0.5 mL/min.

### Measurement of Weight of Total Lipids

The weight of total lipids was determined using the Soxhlet extraction method. It is expressed on total dry weight of the isolated oleosomes.

### Calculation of Lipid Present in Oleosomes

The weight of lipid that is present in the oleosomes of the sunflower oleosome extract or concentrate was calculated by subtracting the amount of free lipid from weight of total lipids of the sunflower oleosome extract or concentrate.

### Measurement of Dry Weight

The percentage dry substance (% DS) of the oleosomes was determined gravimetrically using an MA150 infrared balance (Sartorius). About 2 g of material (i.e; the wet weight (WW)) is applied on an aluminum dish with glass fiber pad. The moisture is evaporated at 105° C. until a stable weight is reached (i.e.; the dry weight (DW)). The percentage dry substance is calculated according to the following formula:

$$\% DS = \frac{D W}{W W} \times 100$$

### Free CGA and Total Free CGAs Measurement

Free chlorogenic acid and total free CGAs were extracted and quantified by HPLC-UV.

### Materials

2-propanol, formic acid and acetonitrile Riddle-de-Haen MS grade were purchased from Thermo Fisher (Geel, Belgium). Dichloromethane LV-GC grade was purchased from Biosolve (Valkenswaard, The Netherlands). Chlorogenic acid; neochlorogenic acid; cryptochlorogenic acid; 3,4-dicaffeoylquinic acid; 3,5-dicaffeoylquinic and 4,5-dicaffeoylquinic acid analytical grade were purchased from Merck (Hoeilart, Belgium). Deionized water at 18.2 MQ (million ohm-cm) was used throughout.

### Method

Total free chlorogenic acid was extracted from 1 g of the sample with 9 mL 1% volume formic acid aqueous solution and 5 mL of dichloromethane by vortex in 2*5 min at 1250 rpm. The aqueous supernatant was injected on an Acquity H-Class HPLC system from Waters equipped with Chromeleon software (Thermo Scientific). The column used, was a Poroshell EC-C18 100*4.6 mm 2.7 μm (Agilent) operated at 35° C. The mobile phase consisted of 0.2% (v/v) formic acid in water (eluent A) and acetonitrile (eluent B) at a flow rate of 1.2 mL/min. The gradient program was as follows: 5% B during 1 min then to 50% B in 10 min (11 min), column was then flushed and equilibrated. Total run time was 15 min. The injection volume for all samples was 10 μL. Phenolic compounds were monitored separately at 326 nm with a window of 4 nm and a speed of peak width 0.1 s. Quantification was performed against external standard solutions of chlorogenic acid at concentration of 0.1, 0.2, 0.5, 1, 2, 5, 10 and 20 μg/mL. Specific response factors were used to quantify the different other phenolic compounds, i.e. neochlorogenic acid, cryptochlorogenic acid, 3,4-dicaffeoylquinic acid, 3,5-dicaffeoylquinic and 4,5-dicaffeoylquinic acid.

### Color Stability Evaluation by Accelerated Color Induction Test

### Equipment & Materials

A metal block thermostat (Rotatherm® Liebisch). This equipment allows a precise temperature control (25° C.) and gentle end-over-end mixing (20 RPM). Sartorius Minisart NML hydrophilic syringe filter (0.2 μm) to permit the removal of oleosomes, and obtaining the aqueous phase/serum.

UV-VIS spectrophotometer (SpectraMax® ABS Plus, Molecular devices).

The objective was to evaluate the color stability of oleosome extracts in an accelerated test. A covalent or non-covalent interaction between co-extracted proteins and CGA was induced by incubating samples for 24 h at pH 9 (covalent) or pH 7 (non-covalent) using 1 M of NaOH or 1 M of HCl. Depending on the type of interaction and substrate concentrations/ratios, different colors could be formed (e.g. yellow or green). It is important to highlight that all color reactions (e.g. CGA-protein complexation) take place in the aqueous phase.

The color stability was evaluated by visual observation and by calculation of the difference in absorption at 400 nm and 680 nm between the end and initial sample. Wavelengths at 400 and 680 nm represent the color absorption peaks for violet and orange, respectively. The complementary colors for 400 and 680 nm are yellow and blue, which in fact are the ones that can be visualized by the human eye.

### Method

Samples were poured into capped glass tubes and incubation was performed in a metal block thermostat. After the incubation, an aliquot was taken from each tube and diluted 20 times with distilled water. Afterwards, samples were filtrated to remove the oleosomes, and obtaining the aqueous phase/serum. 200 μL of aqueous filtrate of each sample was pipetted in a 96-well plate and absorbances (Abs) between 400 and 800 nm (visible range) were detected by means of a UV-VIS spectrophotometer. The color stability was evaluated by calculation of the difference in absorption at 400 nm and 680 nm between the initial sample and the incubated sample according to the following formula:

$${ΔAbs}_{i n c u b ated - initial} = {Abs}_{i n c u b a t e d} - {Abs}_{initial}$$

### 2. Obtaining a Sunflower Oleosome Extract

### 2.1. Procedure for Obtaining HO Sunflower Oleosome Concentrate A

### 2.1.1. Obtaining a Sunflower Oleosome Extract

In order to obtain a sunflower oleosome extract, 1000 kg dehulled seeds from high-oleic sunflower (58.7% fat, 21.2% protein) were soaked during 24 hours in deionized water in a ratio of seeds to water of 1:2 at a temperature of 4° C. under continuous gentle stirring to keep the seeds in suspension without breaking them. The soaking water was discarded by sieving (2 mm mesh) and the soaked seeds were washed by resuspending the seeds in deionized water in a ratio of seeds to water of 1:2 at a temperature of 4° C. The washing water was discarded by sieving (2 mm mesh) and the washed seeds were ground with deionized water in a ratio of soaked seeds to water of 1.5:3 using an inline toothed colloid mill (Colloid Type E—PUC100, Probst & Class) at a flow rate 1000 L/h. A slurry of ground sunflower seeds with a dry substance of 20.7 wt. % was obtained. A sodium bicarbonate solution and ascorbic acid solution were added continuously until a concentration of 1.64 g/L of sodium bicarbonate and 1.43 g/L of ascorbic acid was obtained in the slurry. A sodium hydroxide solution at a concentration of 8% was added until a pH of 7.19 and a conductivity of 2500 microSiemens/cm was reached. The slurry was subsequently decanted using a GEA SCD-306 decanter. The cake (solid heavy phase) was discarded. 300 kg of the obtained oleosome extract (light liquid phase) was used for further microfiltration. The composition of sunflower oleosome extract can be seen in Table 1.

TABLE 1
Sunflower oleosome extract

| | |
|---|---|
| pH | 7.19 |
| Dry matter (wt. %) | 11.3 |
| Protein content (wt. %)* | 17.7 |
| Total lipid content (wt. %)* | 82.3 |
| Total free CGAs (mg/kg)* | 22167 |
| Free CGA (mg/kg)* | 18033 |

*expressed on dry matter of the sunflower oleosome extract

### 3. Example According to the Invention

### 3.1. Step i) Fractionating by Means of Microfiltration the Sunflower Oleosome Extract

The obtained sunflower oleosome extract was heated to 50° C. using a plate heat exchanger and collected in the feed tank for the microfiltration unit where it was kept under continuous agitation. The heated sunflower oleosome extract was concentrated by means of microfiltration.

Microfiltration was carried out on a single-stage tubular multi-channel ceramic microfiltration membrane (Membralox, Pall) with a membrane area of 3.8 m² and a pore size of 0.2 μm. The average transmembrane pressures at the inlet and outlet were 0.8 and 1.1 bar respectively and the circulation flow 90 m³/h (5.5 m/s). The average flux throughout the entire microfiltration process was 45 kg/h/m² and kept constant.

The pH of the obtained oleosomes retentate fraction was lowered to pH 7.0 using an 8% HCl solution. An oleosome retentate fraction with a dry substance of 14.39 wt. % was obtained.

The protein content, lipid content and dry matter content of the obtained oleosome retentate fraction was analyzed.

The composition of oleosome retentate fraction can be seen in Error! Reference source not found.

TABLE 2
Oleosome retentate fraction

| | |
|---|---|
| pH | 7.0 |
| Dry matter (wt. %) | 14.39 |
| Protein content (wt. %)* | 15.29 |
| Total lipid content (wt. %)* | 79.22 |
| Total free CGAs (mg/kg)* | 16010 |
| Free CGA (mg/kg)* | 11444 |

*expressed on dry matter of the sunflower oleosome extract

### 3.2. Step ii) Obtaining the Sunflower Oleosome Composition

The oleosome retentate fraction was further processed by repeating 5 times the following procedure on the same microfiltration system: one volume of oleosome retentate fraction was diluted with one volume of deionized water and subsequently concentrated by means of microfiltration until the initial volume was reached again. Subsequently the obtained retentate fraction was concentrated on the same microfiltration system until a dry substance of 20.91 wt. % was reached (see Example according to the invention, table 4). The average transmembrane pressures at the inlet and outlet were 0.8 and 1.1 bar respectively and the circulation flow 90 m³/h (5.5 m/s). The average flux throughout the entire microfiltration process was 45 kg/h/m² and kept constant.

Table 4 also shows the further characteristics of this example (the example according to the invention), including the results for the content of free and total free CGA.

### 4. Comparative Examples

In existing processes for obtaining sunflower oleosome compositions a sunflower oleosome extract is subjected to a centrifugation step, optionally followed by a second centrifugation washing step.

### 4.1. Obtaining a Sunflower Oleosome Extract

In order to obtain a sunflower oleosome extract, 130 kg dehulled seeds from high-oleic sunflower (58.6% fat, 22.1% protein) were soaked during 24 hours in deionized water in a ratio of seeds to water of 1:2 at a temperature of 4° C. The soaking water was discarded by sieving (2 mm mesh) and the soaked seeds were washed by resuspending the seeds in deionized water in a ratio of seeds to water of 1:2 at a temperature of 4° C. The washing water was discarded by sieving (2 mm mesh) and the washed seeds were ground with deionized water in a ratio of soaked seeds to water of 1.5:3 using a toothed colloid mill (MZ-80/R, Fryma) followed by a stone mill (MK180/MZ120, FrymaKoruma) at a flow rate of 42.2 kg/h soaked seeds and 83.8 L/h deionized water fed to the hopper of the first mill. A sodium bicarbonate solution and ascorbic acid solution were added continuously until a concentration of 2.2 g/L of sodium bicarbonate and 2 g/L of ascorbic acid was obtained in the slurry. A sodium hydroxide solution at a concentration of 8% was added until a pH of 6.94 and a conductivity of 2664 microSiemens/cm was reached. A slurry of ground sunflower seeds with a dry substance of 16.64 wt. % was obtained. The slurry was subsequently decanted using a tricanter (Z2E, Flottweg). The cake (solid heavy phase) was discarded. The oleosome extract (light liquid phase) was used for further centrifugation The composition of sunflower oleosome extract can be seen in Table 3.

TABLE 3
Sunflower oleosome extract

| | |
|---|---|
| pH | 7.29 |
| Dry matter (wt. %) | 10.69 |
| Protein content (wt. %) * | 21.60 |
| Total lipid content (wt. %)* | 72.03 |
| Total free CGAs (mg/kg)* | 23014 |
| Free CGA (mg/kg)* | 21753 |

*expressed on dry matter of the sunflower oleosome extract

### 4.2. Centrifugation of the Sunflower Oleosome Extract

The sunflower oleosome extract was centrifuged using a disc stack liquid-liquid separator (Easyscale 10.S, GEA Westfalia) to yield an oleosome-rich light liquid phase, i.e. comparative example 1 (composition shown in Table) and heavy liquid phase which was discarded.

### 4.3. Washing the Sunflower Oleosome Fraction

A part of the oleosome-rich light liquid phase from comparative example 1 was diluted to 15 wt. % dry substance with deionized water, brought to pH 9.5 using an 8% sodium hydroxide solution and centrifuged for a second time using a disc stack liquid-liquid separator (Easyscale 10.S, GEA Westfalia) to yield an oleosome-rich light liquid phase, and a watery heavy liquid phase which was discarded. Afterwards, the washed oleosome-rich light liquid phase was standardized by bringing back the pH to 7 and diluting it to 34.4 wt. % dry substance with deionized water.

The composition can be seen in TTable (comparative example 2).

### 5. Results

TABLE 4
Oleosome compositions

| | Example | | |
|---|---|---|---|
| | according | | |
| | to the | Comparative | Comparative |
| | invention | example 1 | example 2 |
| Dry matter wt % | 20.9 | 32.6 | 34.4 |
| Protein content ** wt % | 12.2 | 13.2 | 6.7 |
| Total lipid content ** wt % | 87.9 | 86.8 | 93.3 |
| Lipids present as | 95.6 | 89.0 | 92.1 |
| oleosomes *** wt % | | | |
| Free lipids *** wt % | 4.4 | 11.0 | 7.9 |
| Free CGA content ** | 128 | 2681 | 588 |
| mg/kg | | | |
| Total free CGAs | 250 | 3702 | 702 |
| content ** mg/kg | | | |
| Protein to free CGA | ~10/1 | ~3/5 | ~4/3 |
| molar ratio | | | |
| Color stability | | | |
| (aqeuous filtrate) | | | |
| Visual inspection of color | Creamy | Creamy | Creamy |
| of initial sample | white | white | white |
| Abs @ 680 nm initial | 0.0258 | 0.0316 | 0.025 |
| sample | | | |
| Abs @ 400 nm of initial | 0.0471 | 0.0822 | 0.0406 |
| sample | | | |
| Visual inspection of color | Creamy | Green | Green |
| after incubation at pH 9.0, | white | | |
| 25° C., 24 h | | | |
| Abs @ 680 nm incubated | 0.0295 | 0.1030 | 0.2035 |
| at pH 9.0, 25° C., 24 h | | | |
| Abs @ 400 nm incubated | 0.0622 | 0.1956 | 0.4978 |
| at pH 9.0, 25° C., 24 h | | | |
| Δ Abs 680 nm | 0.0037 | 0.0714 | 0.1785 |
| Δ Abs 400 nm | 0.0151 | 0.1134 | 0.4572 |
| Visual inspection of color | Creamy | Creamy | Creamy |
| after incubation at pH 7.0, | white | yellowish | yellowish |
| 25° C., 24 h | | | |
| Abs @ 680 nm incubated | 0.0269 | 0.044 | 0.0309 |
| at pH 7.0, 25° C., 24 h | | | |
| Abs @ 400 nm incubated | 0.042 | 0.1498 | 0.0538 |
| at pH 7.0, 25° C., 24 h | | | |
| Δ Abs 680 nm | 0.0011 | 0.0124 | 0.0059 |
| Δ Abs 400 nm | −0.0051 | 0.0676 | 0.0132 |

** expressed on dry matter of the sunflower oleosome composition
*** expressed on total content of lipids

1. A process for obtaining a sunflower oleosome composition, wherein the process comprises the steps of:
i) fractionating by means of microfiltration a sunflower oleosome extract into a retentate fraction and a permeate fraction, and
ii) collecting the retentate fraction, optionally diluting the retentate fraction, and obtaining the sunflower oleosome composition;
further wherein:
a) the sunflower oleosome extract:
a1) has a total free CGAs content of at least 20000 mg/kg, preferably an amount of free CGA of at least 15000 mg/kg, based on the dry weight of the sunflower oleosome extract,
a2) has a weight of total lipids of at least 70 wt. % based on the dry weight of the sunflower oleosome extract, and
a3) comprises oleosomes, wherein at least 80 wt. % of the total lipids is present in oleosomes; and
b) the sunflower oleosome composition:
b1) has a total free CGAs content that is less than the total free CGAs content of a1),
b2) has a weight of total lipids of at least 70 wt. % based on the dry weight of the sunflower oleosome composition, and
b3) comprises oleosomes, wherein at least 80 wt. % of the total lipids is present in oleosomes.

2. The process according to claim 1, wherein the microfiltration is carried out using a membrane filter having a pore size of less than 2.0 micron, preferably less than 1.0 micron, more preferably less than 0.5 micron, even more preferably 0.1 to 0.2 micron.

3. The process according to claim 1, wherein the microfiltration is carried out using a membrane filter that is selected from the group consisting of ceramic membranes, cellulose acetate membranes, polyamides membranes, and polysulphon membranes, and is preferably a ceramic membrane.

4. The process according to claim 1, wherein the microfiltration is carried out using one or more of the following process conditions:
a transmembrane pressure of less than 2 bar, preferably less than 1.5 bar, more preferably less than 1.2 bar, most preferably from 0.7 to 1.15 bar;
a flux in the range of from 10 to 200 kg/h/m², preferably from 20 to 180 kg/h/m², more preferably from 30 to 50 kg/h/m²; and/or
a temperature in the range of 20 to 60° C., preferably 40 to 60° C., more preferably 45 to 55° C.

5. The process according to claim 1, wherein the sunflower oleosome extract has a pH value in the range of pH 3 to 10, preferably from pH 4 to 9, more preferably from pH 5 to 8.

6. The process according to claim 1, wherein the retentate fraction obtained in step i) is subjected to one or more further fractionations by means of microfiltration into one or more further retentate fractions and further permeate fractions, and wherein said one or more further retentate fractions are collected, and optionally diluting the one or more further retentate fractions, to obtain the sunflower oleosome composition.

7. A sunflower oleosome composition comprising lipids and proteins, wherein the sunflower oleosome composition:
I. has a total free CGAs content of less than 20000 mg/kg, preferably an amount of free CGA of less than 15000 mg/kg, based on the dry weight of the sunflower oleosome composition,
II. has a weight of total lipids of at least 70 wt. % based on the dry weight of the sunflower oleosome composition, and
III. comprises oleosomes, wherein at least 80 wt. % of the total lipids is present in oleosomes.

8. The sunflower oleosome composition according to claim 7, wherein the composition has a total protein weight of up to 25 wt. % based on the dry weight of the oleosome composition, preferably from 5 to 25 wt. %.

9. The sunflower oleosome composition according to claim 7, wherein the sunflower oleosome composition:
i has content of total free CGAs from 0.1 to 600 mg/kg, expressed on the dry weight of the sunflower oleosome composition,
ii content of free CGA is a range of from 0.05 to 400 mg/kg expressed on the dry weight of the sunflower oleosome composition.

10. The sunflower oleosome composition according to claim 7, wherein the sunflower oleosome composition:
i has content of total free CGAs from 0.1 to 600 mg/kg, expressed on the dry weight of the sunflower oleosome composition,
ii content of free CGA is a range of from 0.05 to 400 mg/kg expressed on the dry weight of the sunflower oleosome composition,
iii has weight of total lipids of at least 87 wt. % based on the dry weight of the sunflower oleosome composition, and
iv comprises oleosomes, wherein from 93 to 98 wt % of the total lipids is present in oleosomes.

11. A product comprising the sunflower oleosome composition according to claim 7, wherein said product is selected from the group consisting of food products, feed products, pharmaceutical products, personal care products, nutritional compositions and industrial products.

12. The product according to claim 11, being the nutritional composition in spray dried form.

13. A product selected from the group consisting of food products, feed products, pharmaceutical products, personal care products, nutritional compositions and industrial products, and the product is prepared by adding the sunflower oleosome composition according to claim 7, to product ingredients that are not a sunflower oleosome composition.

14. A use of a microfiltration unit to reduce the amount of total free chlorogenic acids, preferably free chlorogenic acid from a sunflower oleosome extract, wherein
a) the sunflower oleosome extract:
a1) has a total free CGAs content of at least 20000 mg/kg, preferably an amount of free CGA of at least 15000 mg/kg, based on the dry weight of the sunflower oleosome extract,
a2) has a weight of total lipids of at least 70 wt. % based on the dry weight of the sunflower oleosome extract, and
comprises oleosomes, wherein at least 80 wt. % of the total lipids is present in oleosomes.

15. The use according to claim 14 wherein a sunflower oleosome composition is obtained and:
b) the sunflower oleosome composition:
b1) has a total free CGAs content that is less than the total free CGAs content of a1),
b2) has a weight of total lipids of at least 70 wt. % based on the dry weight of the sunflower oleosome composition, and
b3) comprises oleosomes, wherein at least 80 wt. % of the total lipids is present in oleosomes.
